(19) United States
(12) Patent Application Publication
(10) Pub. No.: US 2023/0270997 A1
(43) Pub. Date: Aug. 31, 2023

(54) ELECTRODE PAD AND LOW-FREQUENCY THERAPY APPARATUS

(71) Applicant: OMRON HEALTHCARE Co., Ltd., Kyoto (JP)

(72) Inventor: Tomoyuki NISHIDA, Kyoto (JP)

(21) Appl. No.: 18/313,747

(22) Filed: May 8, 2023

Related U.S. Application Data
(63) Continuation of application No. PCT/JP2021/040354, filed on Nov. 2, 2021.

(30) Foreign Application Priority Data
Nov. 13, 2020 (JP) 2020-189247

(51) Int. Cl. A61N 1/04

(52) U.S. Cl. CPC A61N 1/0496

(57) ABSTRACT

An electrode pad is an electrode pad for low-frequency therapy used in contact with a living body. The electrode pad includes a conductive material having moisture permeability and liquid permeability and a fast-drying member having insulating properties provided on a first face. The conductive material includes a first face facing the side opposite to the side where the body surface of a user is located in a contact state for the electrode pad to be in contact with the living body, and a second face facing the body surface of the user in the contact state.

### CROSS-REFERENCE TO RELATED APPLICATIONS

This application is the U.S. national stage application filed pursuant to 35 U.S.C. 365(c) and 120 as a continuation of International Patent Application No. PCT/JP2021/040354, filed Nov. 2, 2021, which application claims priority to Japanese Patent Application No. 2020-189247, filed Nov. 13, 2020, which applications are incorporated herein by reference in their entireties.

### TECHNICAL FIELD

The present disclosure relates to an electrode pad for low-frequency therapy and a low-frequency therapy apparatus.

### BACKGROUND ART

As an electrode pad in the related art, JP 4708447 B (Patent Document 1) discloses an electrode pad in which a gel layer is provided on each of the two faces of a flexible conductor. In a state of being attached to a living body, an insulating backing is adhered to the gel layer provided on the side that does not come into contact with the living body.

### CITATION LIST

### Patent Literature

Patent Document 1: JP 4708447 B

### SUMMARY OF INVENTION

### Technical Problem

In general, when an electrode pad is used as a bioelectrode for monitoring the potential or physiological potential of a living body, the electrode pad is adhered to a surface of the living body in a dry state.

On the other hand, when an electrode pad is used as a low-frequency therapy apparatus, the electrode pad may be adhered to a surface of a living body in a moist state, such as after exercise or bathing. In such cases, the gel layer in contact with the living body swells by absorbing a liquid that is sweat, or hot water adhering to the body surface, or the like, or a vapor emitted from the body. When swelling, the gel layer will become hard to adhere to the living body or easy to break off. In addition, when the gel layer in a state of absorbing liquid or vapor continues to be in contact with the skin, the skin is likely to become swollen or develop a rash.

The present disclosure has been made in view of the problems described above, and the purpose of the present disclosure is to provide an electrode pad with excellent moisture permeability and a low-frequency therapy apparatus including the same.

### Solution to Problem

An electrode pad based on the present disclosure is an electrode pad for low-frequency therapy used in contact with a living body. The electrode pad includes: a conductive material having moisture permeability and liquid permeability, the conductive material including a first face facing a side opposite to a side where a body surface of a user is located in a contact state for the electrode pad to be in contact with the living body, and a second face facing the body surface of the user in the contact state; and a fast-drying member having insulating properties, the fast-drying member provided on the first face.

According to the above configuration, even when a liquid such as sweat, or vapor reaches the conductive material from the living body in a contact state for the electrode pad to be in contact with the living body, the liquid and vapor are released toward the fast-drying member because the conductive material has moisture permeability and water permeability. The fast-drying member dries such liquid and vapor quickly. As a result, liquid or vapor from the living body is more likely to dissipate outward from the fast-drying member, improving moisture permeability of the electrode pad.

The electrode pad based on the present disclosure described above may further include a gel layer with conductivity, the gel layer provided on the second face. In this case, the above gel layer may have hygroscopicity and liquid absorbency.

According to the above configuration, a liquid such as sweat, or vapor, can be absorbed from the living body first by the gel layer, and the liquid or vapor absorbed by the gel layer can be dissipated from the fast-drying member to the outside of the electrode pad through the conductive material having moisture permeability and liquid permeability.

In the electrode pad based on the present disclosure described above, the above gel layer may include a moisture and liquid absorbing structure. The above moisture and liquid absorbing structure may include a plurality of holes penetrating the gel layer in a thickness direction of the gel layer, and the moisture and liquid absorbing structure may be configured to absorb liquid by the plurality of holes.

According to the above configuration, the moisture and liquid absorbing structure provided in the gel layer includes a plurality of holes penetrating the gel layer in the thickness direction of the gel layer, so that liquids or the like can be more absorbed toward the conductive material by capillary action. This further enhances the moisture permeability of the electrode pad.

In the electrode pad based on the present disclosure described above, the above gel layer may include a moisture and liquid absorbing structure. The above moisture and liquid absorbing structure may include a fibrous material or a tubular member mixed within the above gel layer.

According to the above configuration, the moisture and liquid absorbing structure provided in the gel layer includes the fibrous material or tubular members mixed in the gel layer, so that the hygroscopicity of the gel layer can be enhanced and liquid or vapor can easily move from the living body to the conductive material.

In the electrode pad based on the present disclosure described above, the above gel layer includes a contact surface that is in contact with the body surface of the user in the above contact state. In this case, the contact surface may include a groove that crosses a thickness direction of the gel layer and extends to a side face of the above gel layer.

According to the above configuration, the groove extending to the side face of the gel layer allows a liquid such as sweat, or vapor to dissipate from the side face of the gel layer.

In the electrode pad based on the present disclosure described above, a woven fabric, a knitted fabric or a nonwoven fabric, having hygroscopicity and liquid absorbency, may be embedded in the gel layer in a manner to be exposed on a side face of the gel layer.

According to the above configuration, a liquid such as sweat, or vapor can be dissipated from the side face of the gel layer by the woven fabric, the knitted fabric or the nonwoven fabric, having hygroscopicity and liquid absorbency.

A low-frequency therapy apparatus based on the present disclosure includes the above electrode pad.

### Advantageous Effects of Invention

According to the present disclosure, an electrode pad with excellent moisture permeability can be provided.

### BRIEF DESCRIPTION OF DRAWINGS

FIG. 1 is a schematic cross-sectional view of an electrode pad according to a first embodiment.

FIG. 2 is a schematic cross-sectional view of an electrode pad according to a second embodiment.

FIG. 3 is a schematic cross-sectional view of an electrode pad according to a third embodiment.

FIG. 4 is a plan view of a contact surface of a gel layer provided in an electrode pad according to a fourth embodiment.

FIG. 5 is a schematic perspective view of a low-frequency therapy apparatus according to a fifth embodiment.

FIG. 6 is a block diagram of the low-frequency therapy apparatus according to the fifth embodiment.

### DESCRIPTION OF EMBODIMENTS

Embodiments of the present disclosure will be described in detail below with reference to the drawings. Note that in the following embodiments, identical or common components are given the same reference numerals in the drawings, and the descriptions thereof are not repeated. In addition, in the case where a plurality of embodiments exist in the following description, unless otherwise specified, it is originally intended to appropriately combine characteristic portions of the respective embodiments.

### First Embodiment

FIG. 1 is a schematic cross-sectional view of an electrode pad according to a first embodiment. An electrode pad 100 according to the first embodiment will be described with reference to FIG. 1.

The electrode pad 100 is used for a low-frequency therapy apparatus. The electrode pad 100 is used in contact with the surface of a living body. The electrode pad 100 is provided to be adhered to the surface (body surface) of a living body. The electrode pad 100 can, for example, be brought into contact with a site where a muscle has become tense and poor blood circulation, and electrical stimulation is applied to the site to contract or relax the muscle, thereby promoting blood circulation and relieving muscle fatigue.

As illustrated in FIG. 1, the electrode pad 100 includes a conductive material 10, a fast-drying member 20, a gel layer 30, and a snap button 40.

The conductive material 10 has a first face 10a and a second face 10b that are in a mutual front and back relationship. The first face 10a faces the side opposite to the side on which the body surface of a user is located in the contact state in which the electrode pad 100 is brought into contact with the surface of the living body. The second face 10b faces the body surface of the user in the above contact state.

The conductive material 10 has moisture permeability and water permeability. The conductive material 10 is provided with a plurality of holes penetrating or communicating from the first face 10a to the second face 10b.

As the conductive material 10, for example, a conductive metal cloth such as wire mesh, a conductive cloth in which the surface of the fiber cloth is metal-plated, or a conductive sheet in which a plurality of pores are provided, or the like, can be employed.

As the conductive material 10, a nonwoven fabric having conductivity, a film member having high moisture permeability and water permeability in which conductive polymer is dispersed, or a material partially plated on a material having high moisture permeability and water permeability, can also be employed.

As described above, as the conductive material 10, materials that are devised to enhance moisture permeability and water permeability in the member having conductivity, or materials that are devised to enhance conductivity in the member having moisture permeability and water permeability, can be employed appropriately.

The fast-drying member 20 is located on the first face 10a. The fast-drying member 20 can dissipate absorbed liquid and vapor to the outside. Specifically, the fast-drying member 20 primarily dissipates absorbed liquid and vapor from the side opposite to the side on which the conductive material 10 is located with respect to the fast-drying member 20.

The fast-drying member 20 is insulative. Specifically, as the fast-drying member 20, an insulating fabric, a nonwoven fabric or a porous member, can be employed. The fast-drying member 20 may be made of a moisture-absorbing desiccant such as silica gel covered with an insulating member having breathability.

As a method of giving fast-drying properties to fabric and nonwoven fabric, for example, a method of combining fibers of different thicknesses, a method of combining water-absorbing materials with water-repellent materials, a method of configuring fibers so that stitches become loose when they absorb moisture or water, thereby improving breathability, can be employed appropriately.

When combining fibers of different thicknesses, they may be combined in a multilayer structure. In this case, since the density of the fabric is made different depending on the layer to generate the capillary action, the liquid and the vapor can be quickly moved and effectively dissipated to the side opposite to the side where the conductive material 10 is located. This reduces the unpleasant stickiness of liquids such as moisture or sweat attached to the skin.

For example, water-absorbing polyester and water-repellent polyester may be combined as a method of combining water-absorbing materials with water-repellent materials. In this case, the inside of the electrode pad 100 can be kept dry even when sweating a lot. Specifically, for a large amount of sweat exceeding a certain amount, a water-repellent thread placed on the protrusion part of the skin side of the fabric repels the liquid, allowing the liquid such as the repelled sweat to escape to the side of the electrode pad, while the fast-drying function is activated.

For example, a knitted material using acetate fibers can be employed as a fabric and a nonwoven fabric where the stitches become loose when they absorb moisture or water. In such fabrics and nonwoven fabrics, the crimping of the fibers suppresses breathability in the dry state, while in the wet state due to sweating or the like, the fibers stretch and breathability improves.

In addition to the above, fabric and nonwoven fabric configured with ultrafine chemical fibers, fabric and nonwoven fabric configured with a fiber material combining ethylene vinyl alcohol (EVOH) and polyester, or fabric and nonwoven fabric configured with cupra fibers, can be employed.

Fabric and nonwoven fabric configured with ultrafine chemical fibers have an expanded transpiration area and are highly hygroscopic, liquid absorptive, and dissipative. Fabric and nonwoven fabric configured with a fiber material combining ethylene vinyl alcohol (EVOH) and polyester have water-absorbing fast-drying properties and cool contact feeling. Fabric and nonwoven fabric configured with cupra fibers have no skin layer on the surface and are porous, so that they have excellent moisture absorption and desorption properties and can reduce stuffiness and stickiness.

As the porous member, for example, a foam such as polyurethane processed to prevent stuffiness, can be employed. Additionally, the fast-drying member 20 may be a moisture-permeable film with insulating properties or an insulating sheet member with a plurality of holes.

As described above, as the fast-drying member 20, members to dissipate liquid and vapor passing through the conductive material 10 can be appropriately adopted. As the fast-drying member 20, one that has a large surface area for easy evaporation and fast diffusion of liquid by capillary action is preferred.

The snap button 40 is connected to the conductive material 10 to penetrate fast-drying member 20. The snap button 40 is connected substantially to the center of the first face 10a. The snap button 40 is rustproof Instead of the snap button 40, wiring may be connected to the conductive material 10.

The gel layer 30 is provided on the second face 10b. The gel layer 30 is adhesive to the surface of the living body and is also conductive. In addition, the gel layer 30 is hygroscopic and liquid absorptive.

The gel layer 30 is configured with, for example, an acrylic polymer gel or a urethane-based gel containing an electrolyte. The gel layer 30 may also be configured by a hydrogel.

The gel layer 30 has a moisture and liquid absorbing structure 31. In the present embodiment, in the gel layer 30, the moisture and liquid absorbing structure 31 is configured by a plurality of holes 32 penetrating in the thickness direction of the gel layer 30 so as to be capable of absorbing liquid. The thickness direction of the gel layer is parallel to the direction in which the first face 10a and the second face 10b line up in the conductive material 10.

The plurality of holes 32 may be uniformly provided in the gel layer 30. The gel layer 30 may include a sparse region in which the density of the holes 32 is sparse, and a dense region in which the density of the holes 32 is higher than that of the sparse region. For example, the area around the snap button 40 when viewed from the thickness direction of gel layer 30 may be defined as a sparse region, and the area around the sparse region may be defined as a dense region. In this case, contact of liquid and vapor with the snap button 40 can be suppressed to further prevent the snap button 40 from rusting.

As mentioned above, by utilizing the capillary action via providing the plurality of holes 32, it becomes easy to guide the liquid or vapor from the surface of the living body to the conductive material 10 by the hole 32.

The liquid or vapor guided by the conductive material 10 is released toward the fast-drying member 20 because the conductive material 10 has moisture permeability and liquid permeability. The fast-drying member 20 quickly dries such liquids and vapors. As a result, liquid or vapor from the living body is more likely to be dissipated outward from the fast-drying member 20, improving moisture permeability of the electrode pad 100.

### Second Embodiment

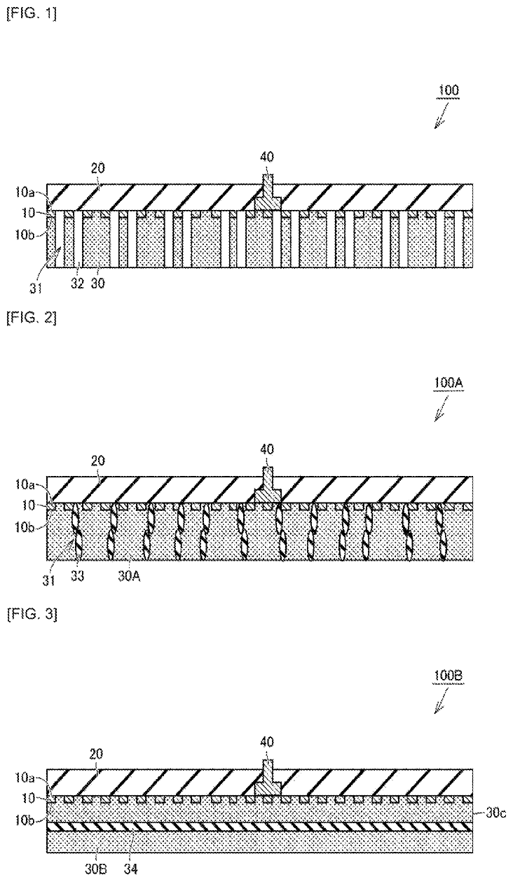

FIG. 2 is a schematic cross-sectional view of an electrode pad according to a second embodiment. An electrode pad 100A according to the second embodiment will be described with reference to FIG. 2.

As illustrated in FIG. 2, the electrode pad 100A according to the second embodiment is different in the configuration of a gel layer 30A when compared with the electrode pad 100 according to the first embodiment. Other configurations are more or less the same.

In FIG. 2, the moisture and liquid absorbing structure 31 of the gel layer 30A is configured by a fibrous material 33 mixed in the gel layer 30A.

The fibrous material 33 can be moved toward the conductive material 10 side while absorbing liquid or vapor absorbed by the gel layer 30A. This further increases the hygroscopicity of the gel layer 30A and allows liquid or vapor to dissipate more quickly to the outside through the conductive material 10 and the fast-drying member 20.

In the above-described description, although the case where the moisture and liquid absorbing structure 31 is configured by the fibrous material 33, has been exemplified, but without limited to this, and the structure may be configured by a tubular member. The tubular member preferably includes an axis that is approximately parallel to the thickness direction of the gel layer 30A. A plurality of tubular members can utilize capillary action to move the liquid or vapor absorbed by the gel layer 30A toward the conductive material 10 side. This further increases the hygroscopicity of the gel layer 30A and allows liquid or vapor to dissipate more quickly to the outside through the conductive material 10 and the fast-drying member 20.

### Third Embodiment

FIG. 3 is a schematic cross-sectional view of an electrode pad according to a third embodiment. An electrode pad 100B according to the third embodiment will be described with reference to FIG. 3.

As illustrated in FIG. 3, the electrode pad 100B according to the third embodiment is different in the configuration of a gel layer 30B when compared with the electrode pad 100 according to the first embodiment. Other configurations are more or less the same.

The gel layer 30B is provided with an embedded layer 34, which is provided to be exposed on a side face 30c included in the gel layer 30B. The embedded layer 34 is configured by a woven fabric, a knitted fabric, or a nonwoven fabric, having hygroscopicity and liquid absorbency, which is embedded in the gel layer 30B so as to be exposed on the side face 30c of the gel layer 30B.

With the above configuration, in addition to the dissipation of the liquid or vapor from the fast-drying member 20 side as in the first embodiment, the embedded layer 34 allows the liquid or vapor to be dissipated from the side face of the gel layer 30B.

### Fourth Embodiment

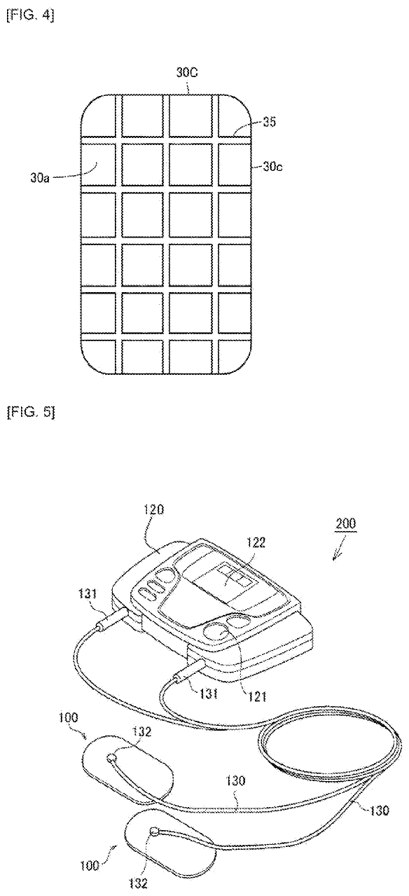

FIG. 4 is a plan view of a contact surface of a gel layer provided in an electrode pad according to a fourth embodiment. The electrode pad according to the fourth embodiment will be described with reference to FIG. 4.

As illustrated in FIG. 4, the electrode pad according to the fourth embodiment is different in the configuration of a gel layer 30C when compared with the electrode pad 100 according to the first embodiment. Other configurations are more or less the same.

In the fourth embodiment, a groove 35 is provided on a contact surface 30a of the gel layer 30C that is in contact with the body surface of the user in the contact state of the electrode pad. The groove 35 is, for example, arranged in a grid. The groove 35 is provided to cross the thickness direction of the gel layer 30C and to extend to the side face of the gel layer 30C.

By providing the groove 35 in this way, in addition to the dissipation of the liquid or vapor from the fast-drying member 20 side as in the first embodiment, the groove 35 allows the liquid or vapor to be moved to the side face of the gel layer 30C. This allows the liquid or vapor to dissipate also from the side face of the gel layer 30C.

The shape of the groove 35 is not limited to a grid shape but may be radial or the like as long as the shape extends to the side face of the gel layer 30C as described above, and an appropriate shape can be employed. The groove 35 may be linear or wavy.

The groove 35 can be applied not only to the first embodiment but also to the second and third embodiments. Furthermore, the groove 35 can be applied to the gel layers without the moisture and liquid absorbing structure 31 as in the first and second embodiments.

### Fifth Embodiment

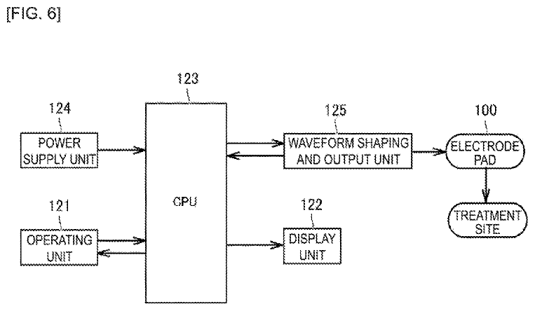

FIG. 5 is a schematic perspective view of a low-frequency therapy apparatus according to a fifth embodiment. FIG. 6 is a block diagram of a low-frequency therapy apparatus according to the fifth embodiment. With reference to FIG. 5 and FIG. 6, a low-frequency therapy apparatus 200 according to the fifth embodiment will be described.

As illustrated in FIG. 5, the low-frequency therapy apparatus 200 includes, schematically, a main body 120 of low-frequency therapy apparatus, a pair of electrode pads 100 to be adhered to a treatment site, and a cord 130.

The main body 120 of low-frequency therapy apparatus includes an operating unit 121 consisting of a plurality of buttons provided for turning the power on and off, various settings, or the like, and a display unit 122 for displaying settings and treatment time. As the main body 120 of low-frequency therapy apparatus, a public one may be employed appropriately.

In the present embodiment, the electrode pad 100 according to the first embodiment is used as the electrode pad, but without limited to this, and the electrode pads of the second to fourth embodiments can be employed appropriately.

The cord 130 electrically connects the main body 120 of low-frequency therapy apparatus to the electrode pad 100. One end of the cord 130 includes a plug 131 to connect to the main body 120 of low-frequency therapy apparatus. The other end of the cord 130 includes a connecting member 132 to connect to the electrode pad 100. The connecting member 132 is, for example, a snap, and is provided so as to be fittable to the snap button 40 of the electrode pad 100.

As illustrated in FIG. 6, the main body 120 of low-frequency therapy apparatus is provided inside with a central processing unit (CPU) 123 that controls the operation of various electronic components, and a waveform shaping and output unit 125 that generates and outputs low-frequency pulses. The CPU 123 is configured to receive electricity from a power supply unit 124 provided in the main body 120 of low-frequency therapy apparatus.

Based on the signal sent from the operating unit 121, the CPU 123 causes the display unit 122 to display the settings, treatment time, or the like, and causes the waveform shaping and output unit 125 to generate low-frequency pulses. The low-frequency pulses output from the waveform shaping and output unit 125 is applied to the treatment site of the human body via the electrode pad 100.

### Other Modified Examples

In the first and second embodiments described above, the case where the gel layer includes the moisture and liquid absorbing structure 31 has been illustrated and described, but without limited to this, and as long as the gel layer has moisture and liquid absorbing properties, the moisture and liquid absorbing structure 31 may be omitted.

In addition, the moisture and liquid absorbing structure 31 is not limited to a plurality of holes, fibrous materials or tubular members mixed in the gel layer, as described in the first to third embodiment, or a woven fabric, a knitted fabric or a nonwoven fabric having hygroscopicity and liquid absorbency embedded in the gel layer, and can be employed appropriately as long as it improves the moisture and liquid absorbing properties of the gel layer. For example, it may be a water-insoluble and hydrophilic material introduced into the gel layer, or a porous material or filler embedded in the gel layer. When a filler is embedded in the gel layer, a void may be created around the filler and capillary action may be generated by the void.

In the first to fourth embodiments described above, the case where the electrode pad 100 includes the gel layer 30 has been illustrated and described, but without limited to this, and the gel layer may be omitted as long as the conductive material 10 allows the current to flow to the living body to relax the muscle. In this case, the electrode pad 100 may be fixed to the surface of the living body by using a fixing member such as a belt.

Supplementary Notes

As described above, the present embodiments include the following disclosures.

Configuration 1

An electrode pad for low-frequency therapy used in contact with a living body, the electrode pad including:
a conductive material (10) having moisture permeability and liquid permeability, the conductive material (10) including:
a first face (10a) facing a side opposite to a side where a body surface of a user is located in a contact state for the electrode pad to be in contact with the living body and a second face (10b) facing the body surface of the user in the contact state; and
a fast-drying member (20) having insulating properties, the fast-drying member (20) provided on the first face (10a).

Configuration 2

The electrode pad according to Configuration 1, further including a gel layer with conductivity, the gel layer provided on the second face (10b), wherein the gel layer has hygroscopicity and liquid absorbency.

Configuration 3

The electrode pad according to Configuration 2, wherein the gel layer (30) includes a moisture and liquid absorbing structure (31), and the moisture and liquid absorbing structure (31) includes a plurality of holes (32) penetrating the gel layer (30) in a thickness direction of the gel layer (30), and the moisture and liquid absorbing structure (31) is configured to absorb liquid.

Configuration 4

The electrode pad according to Configuration 2, wherein the gel layer (30A) includes a moisture and liquid absorbing structure (31), and the moisture and liquid absorbing structure (31) includes a fibrous material or a tubular member mixed within the gel layer (30A).

Configuration 5

The electrode pad according to any one of Configurations 2 to 4, wherein
the gel layer (30C) includes a contact surface (30a) that is in contact with the body surface of the user in the contact state, and
the contact surface (30a) includes a groove (35) that crosses a thickness direction of the gel layer (30C) and extends to a side face (30c) of the gel layer (30C).

Configuration 6

The electrode pad according to any one of Configurations 2 to 5, wherein a woven fabric, a knitted fabric or a nonwoven fabric, having hygroscopicity and liquid absorbency, is embedded in the gel layer (30B) in a manner to be exposed on a side face (30c) of the gel layer (30B).

Configuration 7

A low-frequency therapy apparatus, including the electrode pad according to any one of Configurations 1 to 6.

The embodiments disclosed herein are illustrative in all respects and are not intended as limitations. The scope of the present invention is indicated by the claims and includes all meaning equivalent to the claims and changes within the scope of the claims.

### REFERENCE NUMERALS LIST

10 Conductive material
10a First face
10b Second face
20 Fast-drying member
30, 30A, 30B, 30C Gel layer
30a Contact surface
30c Side face
31 Moisture and liquid absorbing structure
32 Hole
33 Fibrous material
34 Embedded layer
35 Groove
40 Snap button
100, 100A, 100B Electrode pad
120 Main body of low-frequency therapy apparatus
121 Operating unit
122 Display unit
124 Power supply unit
125 Waveform shaping and output unit
130 Cord
131 Plug
132 Connecting member
200 Low frequency therapy apparatus

What is claimed is:

1. An electrode pad for low-frequency therapy used in contact with a living body, the electrode pad comprising:
a conductive material having moisture permeability and liquid permeability, the conductive material including:
a first face facing a side opposite to a side where a body surface of a user is located in a contact state for the electrode pad to be in contact with the living body, and
a second face facing the body surface of the user in the contact state;
a fast-drying member having insulating properties, the fast-drying member provided on the first face; and
a gel layer with conductivity, the gel layer provided on the second face, wherein
the gel layer has hygroscopicity and liquid absorbency,
the gel layer includes a moisture and liquid absorbing structure, and
the moisture and liquid absorbing structure includes a plurality of holes penetrating the gel layer in a thickness direction of the gel layer, and the moisture and liquid absorbing structure is configured to absorb liquid by the plurality of holes.

2. An electrode pad for low-frequency therapy used in contact with a living body, the electrode pad comprising:
a conductive material having moisture permeability and liquid permeability, the conductive material including:
a first face facing a side opposite to a side where a body surface of a user is located in a contact state for the electrode pad to be in contact with the living body, and
a second face facing the body surface of the user in the contact state;
a fast-drying member having insulating properties, the fast-drying member provided on the first face; and
a gel layer with conductivity, the gel layer provided on the second face, wherein
the gel layer has hygroscopicity and liquid absorbency,
the gel layer includes a moisture and liquid absorbing structure, and
the moisture and liquid absorbing structure includes a fibrous material or a tubular member mixed within the gel layer.

3. The electrode pad according to claim 1, wherein
the gel layer includes a contact surface that is in contact with the body surface of the user in the contact state, and
the contact surface includes a groove that crosses the thickness direction of the gel layer and extends to a side face of the gel layer.

4. The electrode pad according to claim 1, wherein
a woven fabric, a knitted fabric or a nonwoven fabric, having hygroscopicity and liquid absorbency, is embedded in the gel layer in a manner to be exposed on a side face of the gel layer.

5. An electrode pad for low-frequency therapy used in contact with a living body, the electrode pad comprising:
a conductive material having moisture permeability and liquid permeability, the conductive material including:
a first face facing a side opposite to a side where a body surface of a user is located in a contact state for the electrode pad to be in contact with the living body, and
a second face facing the body surface of the user in the contact state;
a fast-drying member having insulating properties, the fast-drying member provided on the first face; and
a gel layer with conductivity, the gel layer provided on the second face, wherein
the gel layer has hygroscopicity and liquid absorbency, and
a woven fabric, a knitted fabric or a nonwoven fabric, having hygroscopicity and liquid absorbency, is embedded in the gel layer in a manner to be exposed on a side face of the gel layer.

6. A low-frequency therapy apparatus, comprising the electrode pad according to claim 1.

7. The electrode pad according to claim 2, wherein
the gel layer includes a contact surface that is in contact with the body surface of the user in the contact state, and
the contact surface includes a groove that crosses a thickness direction of the gel layer and extends to a side face of the gel layer.

8. The electrode pad according to claim 2, wherein
a woven fabric, a knitted fabric or a nonwoven fabric, having hygroscopicity and liquid absorbency, is embedded in the gel layer in a manner to be exposed on a side face of the gel layer.

9. The electrode pad according to claim 3, wherein
a woven fabric, a knitted fabric or a nonwoven fabric, having hygroscopicity and liquid absorbency, is embedded in the gel layer in a manner to be exposed on the side face of the gel layer.

10. The electrode pad according to claim 7, wherein
a woven fabric, a knitted fabric or a nonwoven fabric, having hygroscopicity and liquid absorbency, is embedded in the gel layer in a manner to be exposed on the side face of the gel layer.

11. A low-frequency therapy apparatus, comprising the electrode pad according to claim 2.

12. A low-frequency therapy apparatus, comprising the electrode pad according to claim 3.

13. A low-frequency therapy apparatus, comprising the electrode pad according to claim 4.

14. A low-frequency therapy apparatus, comprising the electrode pad according to claim 5.

15. A low-frequency therapy apparatus, comprising the electrode pad according to claim 7.

16. A low-frequency therapy apparatus, comprising the electrode pad according to claim 8.

17. A low-frequency therapy apparatus, comprising the electrode pad according to claim 9.

18. A low-frequency therapy apparatus, comprising the electrode pad according to claim 10.
